(19) United States
(12) Patent Application Publication
(10) Pub. No.: US 2023/0053474 A1
(43) Pub. Date: Feb. 23, 2023

(54) MEDICAL CARE SYSTEM FOR ASSISTING MULTI-DISEASES DECISION-MAKING AND REAL-TIME INFORMATION FEEDBACK WITH ARTIFICIAL INTELLIGENCE TECHNOLOGY

(71) Applicants: Taichung Veterans General Hospital, Taichung City (TW); Tunghai University, Taichung City (TW); Advantech Co.,Ltd., Taipei City (TW)

(72) Inventors: Chieh-Liang WU, Taichung City (TW); Chen-Tsung KUO, Taipei City (TW); Lai-Shiun LAI, Taichung City (TW); Wen-Cheng CHAO, Taichung City (TW); Win-Tsung LO, Taichung City (TW); Ruey-Kai SHEU, Taichung City (TW); Lun-Chi CHEN, Taichung City (TW); Kai-Chih PAI, Taichung City (TW); Jui-Ping CHENG, KeeLung City (TW); Wei-Li CHANG, Taipei City (TW)

(21) Appl. No.: 17/590,174

(22) Filed: Feb. 1, 2022

(30) Foreign Application Priority Data
Aug. 17, 2021 (TW) 110130364

(51) Int. Cl. G16H 50/20; G16H 50/30; G16H 10/60; G06N 5/04

(52) U.S. Cl. CPC G16H 50/20; G16H 50/30; G16H 10/60; G06N 5/04

(57) ABSTRACT

A medical care system for assisting multi-diseases decision-making and real-time information feedback with artificial intelligence technology provided by the invention is capable of obtaining N training models respectively by mathematically operating M different diseases correspondingly based on a batch of collected medical information, obtaining inference results related to at least two diseases by inputting a single patient's data into all or part of the training models to perform mathematical calculation, at the same time, receiving feedback from professionals on the inference results to effectively integrate objective medical data of the patient with subjective medical data of the professionals, and constructing a multi-diseases data model based on the integrated data to be used as a tool for assisting multi-diseases decision-making.

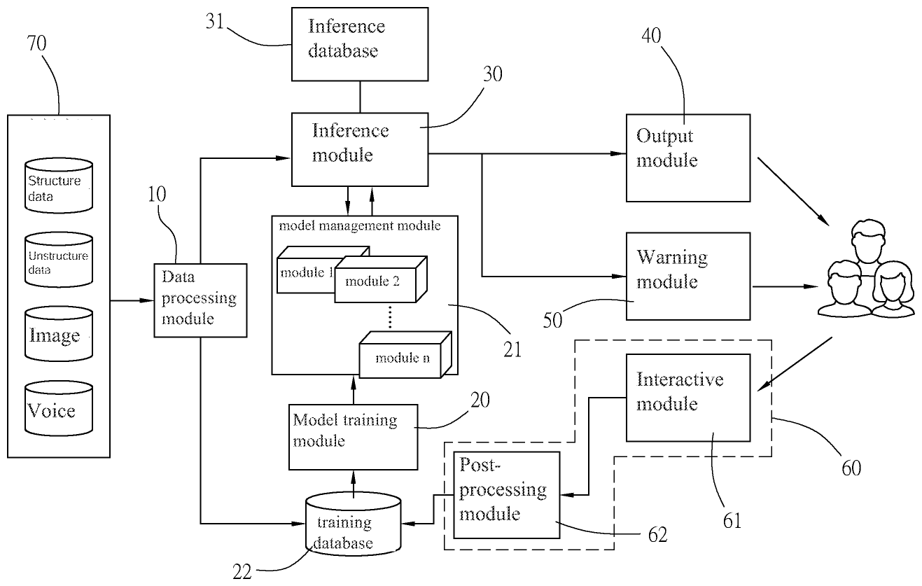

### BACKGROUND OF THE INVENTION

### Field of Invention

The invention is related to medical care, and more particularly refers to a medical care system for assisting multi-diseases decision-making and real-time information feedback with artificial intelligence technology.

### Related Art

The clinical decision support system (CDSS) is capable of making simple treatment decisions based on the clinical information entered by the user for medical care staff to follow or make decisions. Wherein the system operates mainly based on rule-based judgment, and its rules include clinical guidance, medical evidence, and instruction principles derived from medical science.

However, the CDSS still encounters practical obstacles, for example, medical complexity (symptom, family history, gene, epidemiology, relevant medical literatures, etc.) has led to difficulties in system mathematical operation and design. Moreover, thousands of clinical studies are published every year, in addition to the huge amount of data, there are also a lot of contradictory research results, which make system integration and maintenance difficult.

In view of the above problems, how to integrate diverse data in real time and improve an accuracy of disease prediction results will be subjects that the relevant industry needs to ponder and consider.

### SUMMARY OF THE INVENTION

A main object of the invention is to provide a medical care system for assisting multi-diseases decision-making and real-time information feedback with artificial intelligence technology capable of integrating medical data of a patient's clinical manifestations and professional evaluation data from medical care staff, and constructing a data model of a variety of different diseases based on the integrated data to achieve an object of inferring multiple diseases at the same time.

Another object of the invention is to provide a medical care system for assisting multi-diseases decision-making and real-time information feedback with artificial intelligence technology capable of automatically capturing and cleaning up medical information in an external database to be used as data sources required for establishing an initial model of different diseases, without having to manually input or compare data, in addition to saving a great deal of personnel costs, an accuracy of predictions can also be improved by using huge amount of data for mathematical operation.

In order to achieve the above objects, the medical care system for assisting multi-diseases decision-making and real-time information feedback with artificial intelligence technology disclosed in the invention comprises a data processing module, a model training module, an inference module, a model management module and a feedback module; with composition of the above modules, the medical care system is capable of processing a large amount of patients' medical information and/or feedback information from a medical care staff for each patient's status using non-manual methods, and establishing at least one training model for at least two diseases at the same time to be used as a tool to assist the medical care staff in judging multiple diseases of patients, and capable of receiving feedback information from professionals in real time to ensure an accuracy of the medical care system disclosed in the invention.

In one embodiment of the invention, the data processing module collects a medical information of a patient from at least one external database, including text and non-text data, such as image data, audio data, etc., and further processes the patient's medical information to produce a first modeling data and an inference data; wherein:

the first modeling data is a result of processing medical information of a plurality of patients within a predetermined period; and

the inference data is a result of processing medical information of a single patient within a predetermined time range.

The model training module receives a training data in batches and then starts a training procedure, the training procedure performs mathematical calculation for M diseases respectively to establish N training models, and analyzes disease prediction results of each of the training models, when the disease prediction results of any one of the training models do not meet a predetermined standard, the model training module restarts the training procedure and re-establishes the N+1th training model; wherein:

the training data comprises the first modeling data and/or a second modeling data of each batch;

the predetermined standard is used to judge quality of the disease prediction results, such as prediction accuracy, sensitivity, specificity, and clinical experience feedback from clinicians, medical professionals or other professionals;

M is a positive integer greater than 2; and

N is a positive integer greater than 1.

The inference module receives and transmits the inference data and an inference result corresponding to the inference data, wherein the inference result is related to at least two diseases.

The model management module receives all the training models from the model training module and the inference data, selects an inference model from the training models, and performs mathematical operation on the inference data with the inference model to obtain the inference result.

The feedback module receives and analyzes the feedback information from a professional on the inference result, when the feedback information comprises incorrect content of the inference result, the feedback module generates the second modeling data based on the feedback information, wherein, the professional can be a clinician, a medical professional, an information professional, a data processing professional, or one who helps to increase an accuracy of the training models.

In another embodiment of the invention, the model training module further comprises a model internal structure for analyzing the training model of an Xth disease, that is, the model internal structure will obtain a target result set for the Xth disease and a disease prediction result obtained by a Yth training model for the Xth disease, and compare the target result with the disease prediction result, when the comparison result is lower than the predetermined standard, it represents judgment of the Yth training model for the Xth disease should be adjusted, and the model training module establishes the N+1th training model with the model internal structure and its preset weight value; wherein:

X is a positive integer greater than 1; and X is less than or equal to M; and

Y is a positive integer greater than 1, and X is less than or equal to N.

In another embodiment of the invention, the medical care system for assisting multi-diseases decision-making and real-time information feedback with artificial intelligence technology disclosed in the invention further comprises a warning module that receives and judges the inference result from the inference module, when the inference result comprises content that does not meet a normal standard value of the disease corresponding to the inference result, the warning module will display a warning message corresponding to the disease.

In one embodiment, the medical care system for assisting multi-diseases decision-making and real-time information feedback with artificial intelligence technology disclosed in the invention further comprises an output module that receives and displays the inference result from the inference module, specifically, the output module has a display unit that displays the inference result presented in a predetermined format.

In one embodiment, the feedback module further comprises an interactive module for receiving a feedback information input by the medical care staff, and a post-processing module for receiving and processing the feedback information from the interactive module to produce the second modeling data, and the second modeling data will be used as a part of the training data to enable the model training module to calibrate each of the training models, so as to maintain or improve an accuracy of mathematical operational results for predicting diseases.

Wherein, the interactive module further comprises an input unit for the medical care staff to input the feedback information.

In another embodiment of the invention, in order to improve an efficiency of unifying data from different sources, the medical care system for assisting multi-diseases decision-making and real-time information feedback with artificial intelligence technology disclosed in the invention further comprises a training database that receives the first modeling data and the second modeling data of each batch and consolidates the first modeling data and the second modeling data into the training data; wherein, the training database further comprises a storage unit for storing the training data.

In another embodiment, the data processing module uses an information processing procedure to process the received patients' medical information, wherein the information processing procedure distinguishes the medical information from the patients based on similarity or relevance in nature, and compensates values or deletes exceptional values.

In one embodiment, the inference module further comprises an inference database for receiving and storing the inference result.

### BRIEF DESCRIPTION OF THE DRAWINGS

The sole figure is a block diagram of a medical care system according to a preferred embodiment of the invention.

### DETAILED DESCRIPTION OF THE INVENTION

First of all, the terms mentioned in this specification need to be explained as follows.

The terms “mathematical calculation” and “algorithm” mentioned in the invention refer to a program capable of comparing and calculating the input data, and the program refers to the use of various applicable statistical analysis and artificial intelligence algorithms and devices, such as regression analysis method, hierarchical analysis method, cluster analysis method, neural network algorithm, genetic algorithm, machine learning algorithm, deep learning algorithm of various statistical analysis and artificial intelligence algorithms.

The term “medical information” mentioned in the invention refers to information related to a patient's personal and physical state, including personal data of the patient, such as gender, age; information obtained through instrument testing or consultation, such as image records, physical examination results, diet records; information collected by instrument, such as gait, voice, heart beat; information provided by patients or their caregivers; and information provided by medical care staff, such as diagnosis results, prognostic status.

The term “professional” mentioned in the invention refers to a personnel with medical professionalism, data processing professionalism, information professionalism, computer system professionalism, or any other professional in medical information processing system or artificial intelligence for judging medical information, such as clinician, nurse, pharmacist, information engineer, system developer.

Please refer to the sole figure for a medical care system for assisting multi-diseases decision-making and real-time information feedback with artificial intelligence technology provided in a preferred embodiment of the invention mainly comprising a data processing module 10, a model training module 20, a model management module 21, a training database 22, an inference module 30, an inference database 31, an output module 40, a warning module 50, and a feedback module 60, and the above-mentioned modules are connected to each other in a wired or wireless manner, for example, the wired communication method is Ethernet, optical fiber network, and the wireless communication method is 4G, 5G, WIFI, Bluetooth, NFC or RFID.

The data processing module 10 collects a batch of medical information of at least one patient from at least one external database 70, and processes the medical information by an information processing procedure to generate a first modeling data and an inference data respectively, wherein:

the information processing procedure comprises cleaning up data and/or mathematical operation of data, wherein, cleaning up data refers to distinguishing the patient's medical information based on similarity or relevance in nature, and compensating values or deleting exceptional values; and mathematical operation of data refers to calculating the patient's medical information by using expressions such as adding up values, averaging, calculating the median;

the first modeling data refers to the data processing module 10 processing the patients' medical information in batches from the external database 70 within a predetermined period according to a command, and the medical information comprises diagnosis results, prognostic results; and the inference data is a result of processing a single patient's medical information within a predetermined time range to be used as a data source for evaluating or predicting the patient's health status.

Generally speaking, the predetermined period can use year as a unit, and the predetermined time range can use day, hour, minute or second as a unit, for example, the predetermined period is from 2002 to 2012, and the predetermined time range refers to the previous 7 days in which data is collected at 9 a.m. every Monday.

The external database 70 can be, but is not limited to, Hospital Information System (HIS) database, Nursing Information System (NIS) database, or Picture Archiving and Communication System (PACS) of a hospital information room; and data types of the medical information should be classified according to their natures, such as structured data, unstructured data, image data and audio data.

The data processing module 10 is connected to the external database 70 in a wireless communication mode such as 4G, 5G, WIFI, Bluetooth, NFC or RFID, or in a wired transmission mode; and the data processing module 10 accepts data exchange technologies (Extensible Markup Language (XML), JavaScript Object Notation (JSON), CSV) from different computing devices (such as server, personal computer, mobile device), different operating systems (iOS, Android, Windows, UNIX, LINUX) and of different formats, and uses services provided by cross-platform service architecture constructed by related programming languages (such as HTML/HTML5, CSS, JavaScript, PHP, ASP, JSP, C, C++, Java, Object C, Perl, Tcl, PHP, Ruby, Python); however, the technical content of such cross-platform data exchange belongs to the scope of conventional technology, and therefore it will not be further explained here.

The model training module 20 receives a training data in batches, and starts a training procedure to perform mathematical calculation for M diseases to establish N training models, and analyzes disease prediction results of each of the training models, when the disease prediction results of any one of the training models do not meet a predetermined standard, the model training module 20 restarts the training procedure to establish the N+1th training model; wherein, the training data comprises the first modeling data and/or a second modeling data of each batch; M is a positive integer greater than 2; and N is a positive integer greater than 1.

The data processing module 10 obtains and processes a large amount of medical data related to the M diseases in a predetermined interval, for example from 2015 to 2020, from the external database 70, and then generates the first modeling data; the model training module 20 receives the first modeling data, and then performs mathematical calculation for the M diseases, and constructs the N training models related to the M diseases.

The model training module 20 can be, but is not limited to, using algorithms of recursive neural network (RNN), long short-term memory (LSTM) network, or convolutional neural network (CNN) to obtain disease characteristics, the training models and an accuracy of the training models related to an Xth disease.

In addition, when N can be equal to M, it means that each disease corresponds to a single training model; when N and M are not equal, it means that the same disease can have multiple training model architectures, for example, the first modeling data comprises data of gender, age, total protein (T-Protein), albumin, globulin, albumin/globulin ratio (A/G ratio), alkaline phosphatase (ALK-P), whether infected with heart disease, stroke, fatty liver. After mathematical calculation is performed by the model training module 20, a single training model may be generated and related to heart disease and kidney disease, or multiple training models may be generated and related to heart disease and kidney disease respectively. Regardless of a quantity of the training models produced, the model training module 20 will provide factors required to complete the mathematical operation of each of the training models, for example, execution of the A training model requires factors of total protein, blood pressure, age, and cardiac ultrasonic waves; execution of the B training model requires factors of gender, blood pressure, weight, liver ultrasonic waves; the factors required to execute each of the training models may partially overlap.

Furthermore, the model training module 20 has an internal model structure for determining whether to restart the training procedure, if the determination result is yes, the training procedure will be restarted; specifically, the model internal structure will obtain a target result set for the Xth disease and a disease prediction result obtained by a Yth training model for the Xth disease, and compare the target result with the disease prediction result, when the comparison result is lower than the predetermined standard, it represents judgment of the Yth training model for the Xth disease should be adjusted, and the model training module 20 establishes the N+1th training model with the model internal structure and its preset weight value; wherein X is a positive integer greater than 1, and X is less than or equal to M; Y is a positive integer greater than 1, and X is less than or equal to N.

Wherein, the target result is obtained from an external setting or obtained through mathematical calculation by the model training module 20, for example, the target result calculated by the Yth training model for the Xth disease is set to an accuracy rate of 90%, or the target result calculated by the Yth training model for the Xth disease is predicted by the model training module 20 to have an accuracy rate of 90%.

Wherein, the model training module 20 can adopt the following judgment methods to analyze whether each of the training models meets the predetermined standard:

(1) prediction accuracy

if a prediction accuracy rate of the Yth training model for the Xth disease is not less than a preset threshold value, the training procedure is ended; if a prediction accuracy rate of the Yth training model for the Xth disease is less than a preset threshold value, the training procedure is repeated until the prediction accuracy rate is not less than the preset threshold value. In this embodiment, the predetermined threshold value is 95% accuracy;

(2) sensitivity and specificity

since an accuracy of the training models corresponding to some diseases is related to parameter of sensitivity or specificity, or the accuracy of the training models will be referred by the clinical expert, the predetermined threshold value comprises a sensitivity range of 80% to 100% and a specificity range of 40% to 95%; and

(3) clinical experience feedback

unsatisfactory or incorrect opinions feedback by professionals on prediction of the Yth training model for the Xth disease.

The inference module 30 receives and transmits the inference data and an inference result corresponding to the inference data, and has an inference database 31 for receiving and storing the inference result, wherein the inference result is related to at least two diseases. The inference database 31 can be, but not limited to phase-change memory (PRAM), static random-access memory (SRAM), dynamic random-access memory (DRAM), flash disk, read-only memory (ROM), random access memory (RANI), disk or optical disc.

The model management module 21 receives all the training models produced by the model training module 20 and the inference data from the inference module 30, selects at least one of the training models suitable for mathematical calculation of the inference data as an inference model, performs mathematical operation on the inference data with the inference model to obtain the inference result, and transmits the inference result to the inference module 30.

Specifically, the model management module 21 is capable of managing all the training models, that is, the model management module 21 selects the inference model from the training models based on content of the inference data, the diseases corresponding to the inference data, or/and factors such as accuracy, effectiveness of the training models, version of the training models.

The output module 40 receives the inference result from the inference module 30 and displays the inference result in a predetermined format to a display unit or an information system, wherein the display unit can be, but not limited to liquid crystal display (LCD), organic light-emitting diode (OLED) display, electronic whiteboard, medical dashboard or other devices that can be recognized by human sense organs; and the information system is a hospital's HIS, NIS, PACS or a medical software.

The warning module 50 receives and judges the inference result from the inference module. When the inference result comprises content that does not meet a normal standard value of the corresponding disease, the warning module 50 will display a warning message. For example, when the warning module 50 determines that the inference result comprises a risk value for suffering from respiratory failure that exceeds the normal standard value, the warning module 50 will output a warning message, so that an immediate reminder can be sent out to a medical professional, such as clinician, nurse for promptly suggesting corresponding treatment methods for the disease. Wherein, the warning message can be, but not limited to voice message, text message, image, program command, driver hardware program. In addition, normal standard values for different diseases are different, for example, normal standard values for hypertension are 130 mmHg for systolic pressure and 80 mmHg for diastolic pressure.

The feedback module 60 comprises an interactive module 61 and a post-processing module 62, wherein:

the interactive module 61 is connected to a system, such as HIS, NIS, PACS, or a terminal device, such as computer, tablet computer, mobile phone, electronic whiteboard or dashboard to receive a feedback information from a professional on the inference result.

The interactive module 61 has an input unit, which can be, but is not limited to mouse, keyboard, touch panel for a professional to input all or part of the feedback information for the inference result after obtaining the inference result. For example, the professional can send the feedback information to the interactive module 61 through application (APP) installed on a mobile phone, web page, text message, email.

The post-processing module 62 receives and processes the feedback information from the interactive module 61 to produce the second modeling data, and after the model training module 20 receives the second modeling data, the model training module 20 can determine whether to restart the training procedure, if the judgment result is to restart the training procedure, the N+1th training model will be produced.

The training database 22 receives the first modeling data and the second modeling data of each batch, and consolidates the first modeling data and the second modeling data into the training data, and stores the training data. Further, the training database 22 has a storage unit for storing the training data, wherein the storage unit can be, but not limited to phase-change memory (PRAM), static random-access memory (SRAM), dynamic random-access memory (DRAM), flash disk, read-only memory (ROM), random access memory (RANI), disk or optical disc.

In summary, the medical care system for assisting multi-diseases decision-making and real-time information feedback with artificial intelligence technology of the invention has the following advantages:

1. the invention is capable of automatically capturing and analyzing medical information in an external database to be used as data sources required for establishing an initial model of different diseases, without having to manually input or compare data, in addition to saving a great deal of personnel costs, an accuracy of predictions can also be improved by using huge amount of data for mathematical operation; and

2. the invention is capable of integrating objective medical data of the patient with subjective medical data of the professionals, and constructing data models of different diseases based on the integrated data for an object of inferring multi-diseases synchronously.

The above-mentioned embodiments are merely used to illustrate the technical ideas and features of the invention, with an object to enable any person having ordinary skill in the art to understand the technical content of the invention and implement it accordingly, the embodiments are not intended to limit the claims of the invention, and all other equivalent changes and modifications completed based on the technical means disclosed in the invention should be included in the claims covered by the invention.

1. A medical care system for assisting multi-diseases decision-making and real-time information feedback with artificial intelligence technology comprising:
a data processing module collecting a medical information of at least one patient from at least one external database, and performing an information processing procedure in batches to produce a first modeling data and an inference data, wherein:
the first modeling data is a result of processing medical information of a plurality of patients within a predetermined period to include a diagnosis result made from at least one data selected from the group consisting of structured data, unstructured data, image data and audio data;
the inference data is a result of processing medical information of a single patient within a predetermined time range;
a model training module receiving a training data in batches and then starting a training procedure for performing mathematical calculation for M diseases to establish N training models, and analyzing disease prediction results of each of the training models, when the disease prediction results of any one of the training models not meeting a predetermined standard, the model training module restarting the training procedure to establish the N+1th training model; wherein:
the training data comprises the first modeling data and/or a second modeling data of each batch;
M is a positive integer greater than 2;
N is a positive integer greater than 1;
an inference module receiving and transmitting the inference data and an inference result corresponding to the inference data, wherein the inference result is related to at least two diseases;
a model management module receiving all the training models from the model training module and the inference data from the inference module, selecting an inference model from the training models, and performing mathematical operation on the inference data with the inference model to obtain the inference result; and
a feedback module receiving and analyzing a feedback information from a professional on the inference result, when the feedback information comprising incorrect content of the inference result, the feedback module generating the second modeling data based on the feedback information.

2. The medical care system for assisting multi-diseases decision-making and real-time information feedback with artificial intelligence technology as claimed in claim 1, further comprising a warning module for receiving and judging the inference result from the inference module, when the inference result comprising content not meeting a normal standard value of the disease corresponding to the inference result, the warning module displaying a warning message corresponding to the disease.

3. The medical care system for assisting multi-diseases decision-making and real-time information feedback with artificial intelligence technology as claimed in claim 1, further comprising an output module for receiving the inference result from the inference module and displaying the inference result in a predetermined format.

4. The medical care system for assisting multi-diseases decision-making and real-time information feedback with artificial intelligence technology as claimed in claim 1, wherein the feedback module further comprises an interactive module for receiving the feedback information input by the medical care staff, and a post-processing module for receiving and processing the feedback information from the interactive module to produce the second modeling data.

5. The medical care system for assisting multi-diseases decision-making and real-time information feedback with artificial intelligence technology as claimed in claim 1, further comprising a training database for receiving the first modeling data and the second modeling data of each batch and consolidating the first modeling data and the second modeling data into the training data.

6. The medical care system for assisting multi-diseases decision-making and real-time information feedback with artificial intelligence technology as claimed in claim 1, wherein the information processing procedure distinguishes the medical information from the patients based on similarity or relevance in nature, and compensates values or deletes exceptional values.

7. The medical care system for assisting multi-diseases decision-making and real-time information feedback with artificial intelligence technology as claimed in claim 1, wherein the model training module comprises a model internal structure that obtains a target result set for an Xth disease and a disease prediction result obtained by a Yth training model for the Xth disease, and compares the target result with the disease prediction result, when the comparison result is lower than the predetermined standard, it represents judgment of the Yth training model for the Xth disease should be adjusted, and the model training module establishes the N+1th training model with the model internal structure and its preset weight value; wherein:
X is a positive integer greater than 1; and X is less than or equal to M; and
Y is a positive integer greater than 1, and Y is less than or equal to N.

8. The medical care system for assisting multi-diseases decision-making and real-time information feedback with artificial intelligence technology as claimed in claim 1, wherein the inference module further comprises an inference database for receiving and storing the inference result.

9. The medical care system for assisting multi-diseases decision-making and real-time information feedback with artificial intelligence technology as claimed in claim 1, wherein the professional is a personnel with medical professionalism, data processing professionalism, information professionalism, computer system professionalism.
